(19) United States
(12) Patent Application Publication
(10) Pub. No.: US 2008/0124407 A1
(43) Pub. Date: May 29, 2008

(54) INHIBITING CYP3A4 INDUCTION

(75) Inventors: David L. Eaton, Mukilteo, WA (US); Kenneth Thummel, Lake Forest Park, WA (US); Changcheng Zhou, New York, NY (US); Theo Bammler, Seattle, WA (US)

Correspondence Address: NIXON PEABODY LLP - PATENT GROUP, 1100 CLINTON SQUARE, ROCHESTER, NY, 14604, US

(73) Assignee: UNIVERSITY OF WASHINGTON, Seattle, WA (US)

(21) Appl. No.: 11/867,299

(22) Filed: Oct. 4, 2007

Related U.S. Application Data
(60) Provisional application No. 60/828,893, filed on Oct. 10, 2006.

(51) Int. Cl. A61K 36/38; A61K 31/26; A61P 9/12; A61K 31/5517; A61K 31/497

(52) U.S. Cl. 424/730; 514/514; 514/220; 514/252.13

(57) ABSTRACT

The present invention is directed to a method of inhibiting CYP3A4 induction. The method involves administering a compound of the following formula:

R₁—X—(CH₂)ₙ—N═C═S

with R₁, X, and n defined herein, binds to a Pregnane X Receptor or Steroid and Xenobiotic Receptor (SXR or NR1I2) under conditions effective to inhibit CYP3A4 gene induction. The present invention also include a method of administering a compound, described herein, together with the CYP3A4 inducer to prevent a loss of efficacy in the subject to whom the CYP3A4 inducer is repeatedly administered. In addition, such compounds can be administered to block the interaction between a CYP3A4 inducer and another drug being administered that is a substrate of CYP3A4.

This application claims the benefit of U.S. Provisional Patent Application Ser. No. 60/828,893, filed Oct. 10, 2006, which is hereby incorporated by reference in its entirety.

This invention was developed with government funding under National Institutes of Health Grant Nos. R01 ES05780, P30 ES07033, RO1 GM63666. The U.S. Government may have certain rights.

### FIELD OF THE INVENTION

The present invention is directed to inhibiting CYP3A4 induction.

### BACKGROUND OF THE INVENTION

Sulforaphane (SFN) is one of the most biologically active phytochemicals in the human diet (FIG. 1A). It is present at high concentrations in some cruciferous vegetables, especially broccoli (Zhang et al., Proc Natl Acad Sci US A 89:2399-403 (1992); Kushad et al., J Agric Food Chem 47:1541-8 (1999)). Epidemiologic and clinical studies have indicated that diets high in cruciferous vegetables protect against a number of cancers, including non-Hodgkin's lymphoma, liver, prostate, cervical, ovarian, lung, and gastrointestinal tract (Pham et al., Mol Cancer Ther 3:1239-48 (2004); Lund, E. Int J Vitam Nutr Res 73:135-43 (2003); Nagle et al., Int J Cancer 106:264-9 (2003); Steinkellner et al., Mutat Res 480-481:285-97 (2001); Murillo et al., Nutr Cancer 41:17-28 (2001)). Numerous studies using animal models and human cells support the putative chemopreventive effects of SFN (Zhang et al., Proc Natl Acad Sci USA 91:3147-50 (1994); Chung et al., Carcinogenesis 21:2287-91 (2000); Conaway et al., Curr Drug Metab 3:233-55 (2002)). For example, SFN treatment reduced 7,12-dimethylbenz(α)anthracene (DMBA)-induced mammary tumors (Zhang et al., Proc Natl Acad Sci USA 91:3147-50 (1994)), inhibited benzo(α)pyrene-induced forestomach tumors in mice (Fahey et al., Proc Natl Acad Sci USA 99:7610-5 (2002)), lowered the formation of colonic aberrant crypt foci in rats (Chung et al., Carcinogenesis 21:2287-91 (2000)), and inhibited cell proliferation of an HT-29 colon cancer cell line (Frydoonfar et al., Colorectal Dis 6:28-31 (2004)). In addition, in a recent study with human hepatocytes in primary culture, it was demonstrated that pretreatment of hepatocytes with 50 μM SFN produced more than a 90% decrease in DNA adduction of the potent hepatocarcinogen, aflatoxin B1 (Gross-Steinmeyer et al., Toxicological Sciences 84(S1): 1495 (2005)).

The mechanism(s) of action of the putative chemopreventive actions of SFN appear to be multifactorial. SFN can induce apoptosis and cell cycle arrest in human cancer cells (Kim et al., Cancer Res 66:1740-50 (2006); Gamet-Payrastre et al., Cancer Res 60:1426-33 (2000)) and is an inhibitor of histone deacetylases (Myzak et al., Faseb J 20:506-8 (2006)). However, most studies have associated the anticancer effects of SFN with the induction of phase II drug metabolism enzymes, especially the glutathione S-transferases (GST) (Talalay et al., Toxicol Lett 82-83:173-9 (1995); Lee et al., Cancer Lett 224:171-84 (2005)). SFN activates the Keap1/Nrf2 transcriptional factor complex that can bind to antioxidant response elements (ARE) and induce a series of detoxification enzymes, such as NAD(P)H:quinone oxidoreductase-1 (NQO1), certain GSTs and UDP-glucuronosyltransferases (UGTs), and other genes involved in antioxidant response (Fang et al., Chemistry 8:4191-8 (2002); Fahey et al., Food Chem Toxicol 37:973-9 (1999); Fahey et al., Proc Natl Acad Sci USA 94:10367-72 (1997); Dinkova-Kostova et al., Free Radic Biol Med 29:231-40 (2000); Gao et al., Proc Natl Acad Sci USA 101:10446-51 (2004); and McWalter et al., J Nutr 134:3499S-3506S (2004)). Interestingly, it has also been reported that SFN down-regulated CYP3A4 transcription and enzyme activity in cultured human hepatocytes, suggesting another mechanism that could also contribute to its anti-cancer effects (Maheo et al., Cancer Res 57:3649-52 (1997)). Indeed, it was confirmed that SFN consistently and dramatically reduced CYP3A4 mRNA content in human hepatocytes (Gross-Steinmeyer et al., Toxicological Sciences 84(S1): 1495 (2005)). CYP3A4 is among the most important enzymes of the CYP family, because it contributes to the metabolism of more than 50% of clinically used drugs and a corresponding number of xenobiotic chemicals (Guengerich, F. P., Annu Rev Pharmacol Toxicol 39:1-17 (1999)). Induction or inhibition of CYP3A4 is a common cause of adverse drug-drug interactions, which are a major public health problem in the U.S. Adverse drug reactions account for 10-17% of the medical indications for acute hospital admission of elderly patients (Beard, K., Drugs Aging 2:356-67 (1992)) and may contribute to more than 100,000 deaths in the U.S. each year. Moreover, by some estimates it represents the 4^{th} to 6^{th} leading cause of death in the U.S. (Wrighton et al., in Metabolic Drug Interactions (Lippincott Williams & Wilkens, Philadelphia), pp. 115-134 (2000); Flockhart et al., Arch Intern Med 162:405-12 (2002)).

The present invention is directed to overcoming some of these problems in the art.

### SUMMARY OF THE INVENTION

The present invention is directed to a method of inhibiting CYP3A4 induction. This involves administering a compound of the following formula:

R₁—X—(CH₂)ₙ—N═C═S

where:
R₁ is a C₁ to C₄ alkyl group,
X is S═O or O═S═O, and
n is an integer of 2-5,
   that binds to a Pregnane X Receptor or Steroid and Xenobiotic Receptor (SXR or NR1I2) under conditions effective to inhibit CYP3A4 gene induction.

The present invention also relates to a method of preventing a loss of efficacy of a drug that is a substrate of CYP3A4 in a subject that is repeatedly administered a CYP3A4 inducer. This involves administering to the subject a compound of the following formula:

R₁—X—(CH₂)ₙ—N═C═S

where:
R₁ is a C₁ to C₄ alkyl group,
X is S═O or O═S═O, and
n is an integer of 2-5,
   together with the CYP3A4 inducer under conditions effective to prevent a loss of efficacy of a drug that is a substrate of CYP3A4 in a subject that is repeatedly administered the CYP3A4 inducer.

Another aspect of the present invention relates to a method of preventing a loss of efficacy of a drug that is both a CYP3A4 inducer and CYP3A4 substrate in a subject to whom the drug is repeatedly administered. This method involves administering to the subject being treated with the CYP3A4 inducer and the substrate a compound of the following formula:

R₁—X—(CH₂)ₙ—N═C═S

where:
R₁ is a C₁ to C₄ alkyl group,
X is S═O or O═S═O, and
n is an integer of 2-5,
   under conditions effective to prevent a loss of efficacy of the drug that is both a CYP3A4 inducer and CYP3A4 substrate in a subject to whom the drug is repeatedly administered.

The nuclear hormone receptor, steroid and xenobiotic receptor (SXR) (Blumberg et al., Genes Dev 12:3195-205 (1998), which is hereby incorporated by reference in its entirety) (also known as pregnane X receptor (PXR) (Kliewer et al., Cell 92:73-82 (1998), which is hereby incorporated by reference in its entirety), PAR (Bertilsson et al., Proc Natl Acad Sci USA 95:12208-13 (1998), which is hereby incorporated by reference in its entirety), and NR1I2), plays a central role in the transcriptional regulation of CYP3A4 (reviewed in (Kliewer et al., Endocr Rev 23:687-702 (2002); Dussault et al., Crit Rev Eukaryot Gene Expr 12:53-64 (2002), which are hereby incorporated by reference in their entirety). SXR is activated by a diverse array of pharmaceutical agents including taxol, rifampicin (RIF), carbamazepine, SR12813, clotrimazole, phenobarbital, the herbal antidepressant St John's Wort, and peptide mimetic HIV protease inhibitors, such as ritonavir (Kliewer et al., Endocr Rev 23:687-702 (2002); Dussault et al., Crit Rev Eukaryot Gene Expr 12:53-64 (2002), which are hereby incorporated by reference in their entirety). These studies indicate that SXR functions as a xenobiotic sensor (Blumberg et al., Genes Dev 12:3195-205 (1998), which is hereby incorporated by reference in its entirety), to coordinately regulate drug clearance in the liver and intestine via transcriptional regulation of xenobiotic detoxifying enzymes and transporters such as CYP3A4 and p-glycoprotein (ABCB1 or MDR1) (Kliewer et al., Endocr Rev 23:687-702 (2002); Dussault et al., Crit Rev Eukaryot Gene Expr 12:53-64 (2002), which are hereby incorporated by reference in their entirety). Because SFN significantly inhibited CYP3A4 expression, it was tested whether inhibition of CYP3A4 gene expression by SFN is mediated by SXR. Here, applicants show that SFN is a specific antagonist of SXR and it inhibits SXR-mediated induction of numerous genes that regulate drug clearance, including but not limited to CYP3A4 and ABCB1. SFN was able to efficiently inhibit SXR activities and SXR-mediated transcription in a concentration dependent manner. SFN bound directly to SXR and inhibited SXR-coactivator interactions. Furthermore, SFN inhibited SXR-mediated CYP3A4 expression and CYP3A4-mediated midazolam (MDZ) clearance in human primary hepatocytes. Thus, SFN is the first identified relatively non-toxic, naturally occurring antagonist for SXR. This discovery could lead to the development of important new therapeutic and dietary approaches to reduce the frequency of adverse drug reactions.

Rifampicin and other drugs that induce CYP3A4 via SXR-mediated transcriptional activation are frequently associated with adverse drug-drug interactions. Because SFN is able to block the induction of CYP3A4, co-administration of SFN with rifampicin and/or other SXR-agonist drugs could greatly reduce or eliminate the drug-drug interaction associated with SXR-mediated induction of CYP3A4 and other drug metabolizing enzymes or transporters that are regulated, wholly or in part, by SXR. Further, co-administration of SFN with drugs, such as carbamazepine, that are both inducers of CYP3A4 via the SXR-transcriptional activation pathway and serve as substrates for CYP3A4 could allow for a more stable, predictable dosing regimen, and require less drug to achieve the same therapeutic benefit. This could be important for expensive drugs that are both SXR agonists and substrates for CYP3A4, such as some of the antiretroviral drugs used to treat HIV/AIDS. By blocking CYP3A4 induction, it is possible that a smaller amount of drug would have the same efficacy as a higher dose, thereby reducing costs and ensuring efficacious drug use.

### BRIEF DESCRIPTION OF DRAWINGS

FIGS. 1A-E depict efficient SFN inhibition of SXR activity. FIG. 1A shows the structure of SFN (4-methylsulfinylbutyl isothiocyanate). FIG. 1B shows HepG2 cells transiently transfected with full-length SXR together with a CYP3A4-luc reporter and CMX-β-galactosidase transfection control plasmid. After transfection, cells were treated with control medium or medium containing 10 μM RIF or RU486 in the absence or presence of SFN at the indicated concentrations for 24 hrs. Results were presented as relative luciferase units (R.L.U.) normalized to the β-galactosidase internal control. FIG. 1C shows HepG2 cells transiently transfected as described above. Cells were treated with 10 μM RIF with indicated concentrations of SFN for 24 hrs. FIG. 1D shows HepG2 cells transiently transfected with GAL4-SXR, a GAL4 reporter fused to luciferase and CMX-β-galactosidase transfection control plasmids. Cells were then treated with 10 μM RIF and SFN at the indicated concentrations for 24 h. FIG. 1E shows HepG2 cells co-transfected with GAL4 reporter and a series of GAL4 constructs in which the GAL4 DNA binding domain is linked to the indicated nuclear hormone receptor ligand binding domain (LBD). Cells were treated with the appropriated ligand or ligand plus SFN (10 μM). The ligands used were mouse PXR (10 μM PCN), rat PXR (10 μM PCN), VDR (10 nM 1,25(OH)₂D₃), and CAR (1 μM CITCO), PPARα (10 μM WY-14643), PPARγ (10 μM Troglitazone), and RXR (100 nM 9-cis-retinoic acid). Results in the presence of SFN are presented as percent activation relative to the normalized luciferase values in the presence of ligands (100%).

FIG. 2 depicts SFN specifically bound to the purified SXR ligand binding domain. His₆-SXR LBD was co-expressed with the SRC-1 receptor interaction domain and purified. The receptor complex was bound to nickel chelate FlashPlates and incubated with 50 nM of ³H-SR12813 in the presence of the indicated concentration of SFN or clotrimazole. Values represent the average of triplicates ±S.E.

FIG. 3 depicts SFN inhibiting SCR co-activator interactions. HepG2 cells were transfected with a GAL4 reporter and VP16-SXR as well as an expression vector for GAL4 DNA-binding domain or GAL4 DNA-binding domain linked to the receptor interaction domains of the indicated SXR co-activators (GAL-SRC1, GAL-PBP, GAL-ACTR or GAL-TIF2). Cells were then treated with control medium or medium containing 10 μM RIF in the presence or absence of SFN at indicated concentrations.

FIG. 4 A-D depict SFN inhibition of SXR-mediated CYP3A4 expression in human primary hepatocytes and LS180 cells. FIGS. 4A and 4B show human primary hepatocytes from two different donors, and FIG. 4C shows LS180 human intestinal epithelial cells pre-treated 24 hrs. with 10 or 25 μM SFN before addition of 10 μM RIF or RU486 for 24 hrs, as indicated. FIG. 4D shows human primary hepatocytes and LS180 cells were treated with 10 or 25 μM SFN for 48 h. Human primary hepatocytes were obtained from LTPADS (Liver Tissue Procurement and Distribution System, Pittsburg, Pa.) as attached cells in 6-well plates. Total RNA from each sample was isolated and the expression of indicated genes was determined by Quantitative Real-time PCR (“QRT-PCR”).

FIG. 5 depicts SFN inhibition of CYP3A4-mediated midazolam (MDZ) clearance in human primary hepatocytes. Midazolan is a CYP3A-specific substrate and allows estimation of CYP3A4 activity. Human primary hepatocytes were pre-treated with 10 or 25 μM of SFN for 24 hrs before addition of 10 μM RIF. After 24 hrs, cells were rinsed with PBS and then incubated with 8 μM MDZ for 6 hrs. Supernatant media was collected and 1'OH-MDZ concentration was measured by LC-MS.

FIG. 6 depicts structural determinants of SXR antagonism by SFN. HepG2 cells were transiently transfected with full-length SXR together with a CYP3A4-luc reporter and CMX-β-galactosidase transfection control plasmid. After transfection, cells were treated with control medium or medium containing 10 μM RIF in the absence or presence of SFN analogs at the indicated concentrations for 24 hrs.

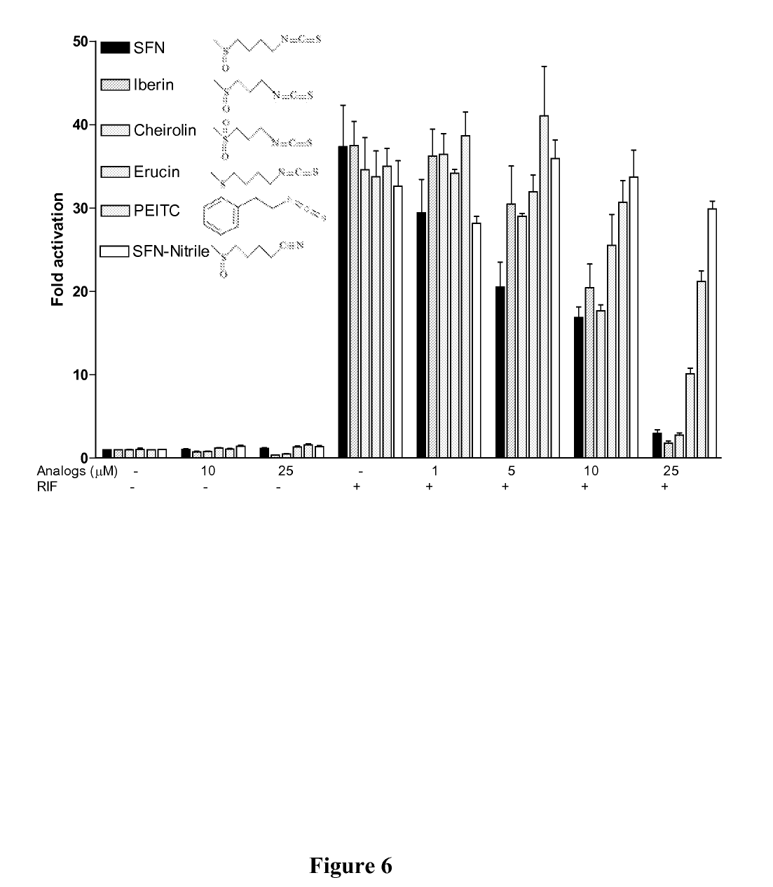

FIG. 7 depicts the effect of 10, 25, and 50 μM SFN on CYP3A4 mRNA levels (normalized to beta actin) in hepatocytes of 10 human donors. Human primary hepatocytes were obtained from LTPADS (Liver Tissue Procurement and Distribution System, Pittsburg, Pa.) as attached cells in 6-well plates. Hepatocytes were treated for 48 hrs. with SFN or vehicle (DMSO) only. Total RNA from each sample was isolated and the expression of indicated genes was determined by QRT-PCR. Values given are relative to 0 μM SFN control.

FIG. 8 depicts hepatocytes from two donors (#11 and #12) treated with Broccoli juice extract containing 2.6 μM (J/SFN 2.6 μM) or 6.6 μM SFN (J/SFN 6.6 μM), or 2.6 μM, 6.6 μM, 10 μM, and 25 μM SFN. Total cellular protein was isolated and CYP3A4 protein levels were determined by immunoblot analysis with specific human CYP3A4 antibody. Values given are relative to DMSO vehicle control.

FIG. 9 depicts primary human hepatocytes from three donors (#11, #12, and #13) treated with Broccoli juice extract containing 2.6 μM (J2.6) or 6.6 μM SFN (J6.6), or 2.6 μM, 6.6 μM, 10 μM, and 25 μM SFN. S9 fractions were prepared and midazolam activities were measured by LC-MS. Mean activities relative to the vehicle controls are displayed.

FIG. 10 depicts the effect of SFN on mRNA expression of a selected list of genes. Human primary hepatocytes isolated from three individual livers were treated for 48 hrs. with 10 or 50 μM SFN. Total RNA from each sample was isolated and mRNA levels were measured by microarray analysis. A “+” or “−” indicates up or down-regulation, respectively, relative to the vehicle control; statistical p values are provided in parentheses.

### DETAILED DESCRIPTION OF THE INVENTION

The present invention is directed to a method of inhibiting CYP3A4 induction. The first step of the method involves administering a compound of the following formula:

R₁—X—(CH₂)ₙ—N═C═S

where:
R₁ is a C₁ to C₄ alkyl group,
X is S═O or O═S═O, and
n is an integer of 2-5,
   that binds to a Pregnane X Receptor or Steroid and Xenobiotic receptor (SXR or NR1I2) under conditions effective to inhibit CYP3A4 gene induction.

In one embodiment of the present invention, the compound has the formula:

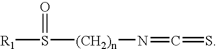

### Preferably, R₁ may be methyl, and n can be 4 in the above compound.

This aspect of the present invention is carried out under conditions effective to additionally inhibit induction of one or more of the following genes: ABCB1 (MDR1), ABCC2 (MRP2), CYP1A2, CYP2B6, CYP2C8, CYP2C9, CYP2C19, CYP3A7, CYP7A1, SULT2A1, UGT1A1, UGT1A3, UGT1A4, PAPSS2, ALAS1, or AHR.

As used above, and throughout the description of the invention, the following terms, unless otherwise indicated, shall be understood to have the following meanings:

The term “alkyl” means an aliphatic hydrocarbon group which may be straight or branched having about 1 to about 6 carbon atoms in the chain. Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

The present invention also relates to a method of preventing a loss of efficacy of a drug that is a substrate of CYP3A4 in a subject that is repeatedly administered a CYP3A4 inducer. This method involves administering to the subject a compound of the following formula:

R₁—X—(CH₂)ₙ—N═C═S

where:
R₁ is a C₁ to C₄ alkyl group,
X is S═O or O═S═O, and
n is an integer of 2-5,
   together with the CYP3A4 inducer under conditions effective to prevent a loss of efficacy of a drug that is a substrate of CYP3A4 in a subject that is repeatedly administered the CYP3A4 inducer.

In carrying out this aspect of the present invention, the subject is desirably a human.

The compounds of the present invention can be administered alone, as indicated above, or utilized as biologically active components in pharmaceutical compositions with suitable pharmaceutically acceptable carriers, adjuvants and/or excipients. The compounds of the present invention may also be formulated with a drug that is an inducer of CYP3A4.

In accordance with the present invention, the compounds and/or corresponding compositions can be introduced via different administration routes, which include orally, parenterally, intravenously, intraperitoneally, by intranasal instillation, or by application to mucous membranes, such as, that of the nose, throat, and bronchial tubes.

The active compounds of the present invention may be orally administered, for example, with an inert diluent, or with an assimilable edible carrier, or they may be enclosed in hard or soft shell capsules, or they may be compressed into tablets.

The quantity of the compound administered will vary depending on the patient and the mode of administration and can be any effective amount. The quantity of the compound administered may vary over a wide range to provide in a unit dosage an effective amount of from about 0.01 to 20 mg/kg of body weight of the patient per day to achieve the desired effect. The amount of active compound in such therapeutically useful compositions is such that a suitable dosage will be obtained. Preferred compositions according to the present invention are prepared so that an oral dosage unit contains between about 1 and 250 mg of active compound.

For example, with oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both.

These active compounds and/or pharmaceutical compositions may also be administered parenterally. Solutions of these active compounds and/or compositions can be prepared in water. Dispersions can also be prepared in glycerol, liquid polyethylene glycols, and mixtures thereof in oils.

Illustrative oils are those of animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the pharmaceutical form of the present invention must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds and/or pharmaceutical compositions of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Some of the compounds of the present invention can be in the form of pharmaceutically acceptable acid-addition and/or base salts. All of these forms of salts are within the scope of the present invention.

Pharmaceutically acceptable acid addition salts of the compounds of the present invention include salts derived from nontoxic inorganic acids, such as hydrochloric acid, nitric acid, phosphoric acid, sulfuric acid, hydrobromic acid, hydroiodic acid, hydrofluoric acid, phosphorous acid, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety).

The acid addition salts of said basic compounds are prepared by contacting the free base forms with a sufficient amount of the desired acid to produce the salt in the conventional manner.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N-dibenzylethylenediamine, chloroprocaine, choline, diethanolamine, dicyclohexylamine, ethylenedianline, N-methylglucamine, and procaine (see, for example, Berge S. M. et al., “Pharmaceutical Salts,” Journal of Pharmaceutical Science, 66:1-19 (1997), which is hereby incorporated by reference in its entirety).

The base addition salts of the acidic compounds are prepared by contacting the free acid form with a sufficient amount of the desired base to produce the salt in the conventional mariner.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

The CYP3A4 inducer may be an anti-cancer drug, an antibiotic, an antiretroviral drug, an antidepressant, a hypolipidemic drug, an anti-epileptic drug, an anti-hypertensive drug, an anti-inflammatory drug, or a wakefulness promoting drug.

Suitable anti-cancer drugs include paclitaxel, topotecan, eptopside, docetaxel, discodermolide, epothilone, vincristine, cyclophosphamide, or tamoxifin.

Useful antibiotics include rifampicin, rifabutin, flucloxacillin, nafcillin, or artemisinin.

Suitable antiretroviral drugs are efavirenz, amprenavir, nevirapine, didanosine, or ritonavir.

The hypolipidemic drug can be avasimibe or guggulsterone.

Exemplary antidepressant drugs include hypericin or St. John's Wort.

A suitable anti-epileptic drug is phenytoin, carbamazepine, topiramate, felbamate, or phenobarbital.

Useful anti-hypertensive drugs include bosentan.

Appropriate anti-inflammatory drugs are dexamethasone, prednisolone, methylprednisolone, or prednisone.

An appropriate drug used to treat narcolepsy and other sleep disorders is modafanil.

Another aspect of the present invention relates to a method of preventing a loss of efficacy of a drug that is both a CYP3A4 inducer and CYP3A4 substrate in a subject to whom the drug is repeatedly administered. This method involves administering to the subject being treated with the CYP3A4 inducer and the substrate a compound of the following formula:

R₁—X—(CH₂)ₙ—N═C═S

where:
R₁ is a C₁ to C₄ alkyl group,
X is S═O or O═S═O, and
n is an integer of 2-5,
   under conditions effective to prevent a loss of efficacy of the drug that is both a CYP3A4 inducer and CYP3A4 substrate in a subject to whom the drug is repeatedly administered.

This aspect of the present invention can be carried out with the compounds, formulations, subjects, and modes of administration described above.

In carrying out this embodiment of the present invention, the other drug is a substrate for CYP3A4.

In accordance with one embodiment of the present invention, the other drug can be an antiepileptic drug, such as carbamazepine, or a psychotropic drug, such as, alprazolam, midazolam, triazolam, buspirone, or ziprasidone.

Alternatively, the other drug can be an anti-hypertensive or cardiovascular drug like nifedipine, felodipine, nicardipine, verapamil, eplerone, lovastatin, atorvastatin, simvastatin, quinidine, or amiodarone.

The other drug can also be an antimicrobial drug such as, clarithromycin, erythromycin, itraconazole, ketocanazole, quinine, amprenavir, saquinavir, or indinavir.

The other drug may be an anticancer drug such as, etoposide, vinicristine, tamoxifen, toremifene, cyclosphosphamide, or ifasfamide.

The other drug may also be a pain medication such as, alfentanyl, sufentanyl, or fentanyl.

Alternatively, the other drug may be an immunosuppressive agent such as, cyclosporine, tacrolimus, or sirolimus.

The other drug can be a contraceptive drug such as, ethinyl estradiol (and its prodrug mesantrol), norethindrone, estrone, estradiol, progesterone, medroxyprogesterone, or the antiprogestin mifepristone.

The other drug may be the antiobesity drug sibutramine or sildenafil, which is a drug used to treat male erectile dysfunction.

### EXAMPLES

### Example 1

### Reagents and Plasmids

SFN, Rifampicin (RIF), mifepristone (RU486), and clotrimazole (CLOT) were purchased from Sigma-Aldrich; PCN, CITCO, WY-14643, Troglitazone, and 9-cis-retinoic acid were purchased from Bio Mol; and 1,25(OH)₂D₃ was purchased from Calbiochem. SXR, GAL4-SXR LBD, VP16-SXR, CMX-β-gal expression vectors; SXR-dependent CYP3A4 promoter reporter (CYP3A4XREM-Luc) and GAL4 reporter (MH100-Luc) have been previously described (Blumberg et al., Genes Dev 12:3195-205 (1998); Synold et al., Nat Med 7:584-90 (2001); Zhou et al, Drug Metab Dispos 32:1075-82 (2001); Zhou et al., J Clin Immunol 24:623-36 (2004); Drocourt et al., Drug Metab Dispos 29:1325-31 (2001), which are hereby incorporated by reference in their entirety).

### Example 2

### Cell Culture

The human intestinal epithelial cell line, LS180, was obtained from American Type Culture Collection and cultured in DMEM containing 10% FBS at 37° C. in 5% CO₂. The cells were seeded into 6-well plates and grown in DMEM-10% FBS until 70-80% confluence. Twenty-four hours before treatment, the medium was replaced with DMEM containing 10% resin-charcoal stripped FBS. Immediately before treatment, the medium was removed; the cells were washed once with PBS and then treated with compounds or DMSO vehicle for appropriate times. Human primary hepatocytes were obtained from LTPADS (Liver Tissue Procurement and Distribution System, Pittsburgh, Pa.) as attached cells in 6-well plates. The hepatocytes were maintained in hepatocyte medium (Sigma-Aldrich) for at least 24 h before treatment.

### Example 3

### Transient Transfection and Luciferase Assay

Cell transfection assays and Luc and β-galactosidase assays were performed as described (Zhou et al., Drug Metab Dispos 32:1075-82 (2001), which is hereby incorporated by reference in its entirety). To test the ability of SFN to inhibit SXR or other nuclear receptors, HepG2 cells were seeded into 12-well plates overnight and transiently transfected with the control or SXR expression plasmid, together with the CYP3A4XREM-Luciferase reporter and CMX-β-galactosidase transfection control plasmids using FuGene 6 (Roche) in serum-free DMEM. Twenty-four hours post-transfection, the cells were treated with DMSO as a negative control, the known SXR ligands RIF, RU486, and clotrimazole, in the absence or presence of SFN. The cells were lysed 24 h after treatment, and β-galactosidase and luciferase assays were performed as described (Grun et al., J Biol Chem 277:43691-7 (2002), which is hereby incorporated by reference in its entirety). Reporter gene activity was normalized to the β-galactosidase transfection controls, and the results were expressed as normalized RLU per OD β-galactosidase per minute to facilitate comparisons between plates. Fold induction was calculated relative to solvent controls. Each data point represents the average of triplicate experiments +/±SEM and was replicated in independent experiments. For mammalian two-hybrid assays, HepG2 cells were transfected with GAL4 reporter, VP16-SXR, and GAL-SRC1, GAL-PBP, GAL-ACTR, or GAL-TIF2 (kindly provided by Dr. B. M. Forman, City of Hope National Medical Institute) (Synold et al., Nat Med 7:584-90 (2001), which is hereby incorporated by reference in its entirety). The cells were then treated with 10 μM RIF or RU486 in the presence or absence of SFN at the indicated concentration.

### Example 4

### Ligand Binding Assays

N-terminal His₆-tagged human SXR ligand binding domain was expressed in Escherichia coli together with the SRC-1 receptor interaction domain and scintillation proximity assays were performed essentially as described (Tabb et al., J Biol Chem 278, 43919-27 (2003); Zhou et al., Drug Metab Dispos 32:1075-82 (2001), which are hereby incorporated by reference in their entirety). Briefly, active protein was refolded from inclusion bodies solubilized in denaturation buffer (6 M guanidium-HCL, 50 mM HEPES pH7.4, 0.2 M NaCl, 25 mM DTT, 1% w/v Triton-X100) by rapid 10-fold dilution into binding buffer (50 mM HEPES pH 7.4, 1 M sucrose, 0.2 M NaCl, 0.1 mM DTT, 0.1% w/v CHAPS) followed by dialysis overnight at 4° C. against binding buffer. Binding assays were performed by coating 96-well nickel chelate FlashPlates (Perkin Elmer Life Sciences) with a 10-fold molar excess of protein for one hour at 22° C. in binding buffer (50 mM Hepes, pH 7.4, 200 mM NaCl, 1 M sucrose, 0.1% CHAPS). Unbound protein was removed from the wells by washing four times with binding buffer. ³H-SR12813 (Amersham-Pharmacia BioSciences) was added to a final concentration of 50 nM in each well, either alone or together with competitor ligands in binding buffer as indicated. Incubation was continued for 3 hours at room temperature. Total counts were measured using a Topcount scintillation counter (Packard, Meriden, Conn.). Counts remaining after the addition of 10 μM clotrimazole were taken as non-specific background and subtracted from all wells. All assays were performed in triplicate and reproduced in independent experiments.

### Example 5

### RNA Isolation and Quantitative Real-time PCR (QRT-PCR) Analysis

Following treatment of primary human hepatocytes or LS180 cells with SFN or solvent, total RNA was extracted using the Trizol reagent (Invitrogen) as recommended by the supplier. Reverse transcription of 2 μg total RNA using oligod(T) 15 primer and Super-script™II RNaseH-(Gibco) was performed as suggested by Gibco. Total RNA was isolated from primary hepatocytes and LS180 cells using TRIzol regent (InVitrogen Life Technology) according to the manufacturer-supplied protocol. Quantitative real time PCR was performed using gene specific primers and the SYBR green PCR kit (Applied Biosystems) in an ABI 7900 system (Applied Biosystems). All samples were quantified using the comparative Ct method for relative quantification of gene expression, normalized to GAPDH (Zhou et al., Drug Metab Dispos 32:1075-82 (2001); Livak et al., Methods 25:402-8 (2001), which are hereby incorporated by reference in their entirety). The following primer sets were used in this study: CYP3A4 (5′-GGCTTCATCCAATGGACTGCATAAAT-3′ (SEQ ID NO:1) and 5′ -TCCCAAGTATAACACTCTACACAGACAA-3′ (SEQ ID NO:2); MDR1 (5′-CCCATCATTGCAATAGCAGG-3′ (SEQ ID NO:3) and 5′-GAGCATACATATGTTCAAACTTC-3′ (SEQ ID NO:4)); UGT1A1(5′-TGCTCATTGCCTTTTCACAG-3′ (SEQ ID NO:5) and 5′-GGGCCTAGGGTAATCCTTCA-3′ (SEQ ID NO:6)); NQO1(5′-GGCAGAAGAGCACTGATCGTA-3′ (SEQ ID NO:7) and 5′-TGATGGGATTGAAGTTCATGGC-3′ (SEQ ID NO:8)); GAPDH (5′-GGCCTCCAAGGAGTAAGACC-3′ (SEQ ID NO:9) and 5′-AGGGGAGATTCAGTGTGGTG-3′ (SEQ ID NO:10)).

### Example 6

### MDZ Clearance Analysis

An internal standard mixture containing ¹⁵N₃-labeled MDZ metabolite, 1′-OH MDZ, was prepared by incubating 6 nmol of cytochrome P450 (using HL-122 microsomes) with 100 μg of ¹⁵N₃-MDZ and 12 mg of NADPH (final concentration, ˜1.5 mM) in potassium phosphate buffer (0.1 M, pH 7.4, in a final volume of 8 ml) at 37° C. After 10 min, the reaction was stopped by the addition of 8 ml of Na₂CO₃ (0.1 M, pH 12). The compounds were extracted twice with 20 ml of ethyl acetate, and the solvent was evaporated to dryness under a stream of nitrogen. The remaining solid was then reconstituted in 20 ml of methanol, split into two 10-ml aliquots, and stored at −80° C. To determine CYP3A4 activity, human primary hepatocytes were pre-incubated with 10 or 25 μM SFN for 24 hrs before addition of 10 μM RIF. Twenty-four hrs later, cells were rinsed with media 3 times and then incubated with new media containing 8 μM MDZ for 6 hrs. The supernatant media were collected for quantitation of 1′-OH MDZ formation, as described before (Paine et al., J Pharmacol Exp Ther 283:1552-62 (1997), which is hereby incorporated by reference in its entirety). Briefly, samples were spiked with 100 μl of a 1:5 dilution of the internal standard mixture, which represented ˜50 ng of ¹⁵N₃-labeled 1′-OH MDZ. The metabolites were extracted with 5 ml of ethyl acetate, the solvent was removed under nitrogen, and the concentrated extracts were dissolved in 100 μl of derivatizing reagent [10% N-methyl-N-(t-butyl-dimethylsilyl)trifluoroacetamide in acetonitrile]. The samples were then transferred to autoinjector vials and were analyzed for 1′-OH MDZ by selective ion gas chromatography-negative chemical ionization mass spectrometry (GC/NCI-MS) as previously described (Paine et al., J Pharmacol Exp Ther 283:1552-62 (1997), which is hereby incorporated by reference in its entirety). The 1′-OH MDZ was quantified by comparing peak area ratios with standard curves prepared by the addition of known amounts of 1′-OH MDZ (0-160 pmol) and 100 μl of internal standard to phosphate buffer.

### Example 7

### SFN Efficiently Inhibits SXR Activity

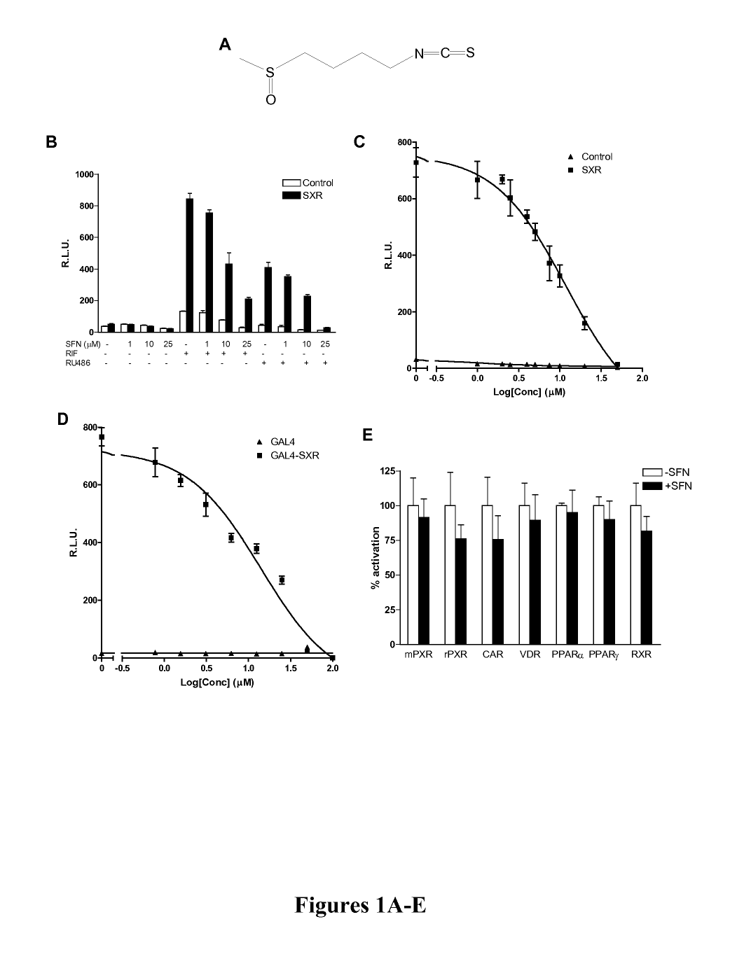

SFN consistently and dramatically reduced CYP3A4 mRNA content in human hepatocytes. SXR contributes substantially to both constitutive and inducible expression of CYP3A4 (Kliewer et al., Endocr Rev 23:687-702 (2002); Dussault et al., Crit Rev Eukaryot Gene Expr 12:53-64 (2002), which are hereby incorporated by reference in their entirety) and several other genes involved in xenobiotic disposition (e.g., ABCB1 (MDR1) (Geick et al., J Biol Chem 276:14581-7 (2001), which is hereby incorporated by reference in its entirety). Importantly, SXR is activated by a diverse array of pharmaceutical agents, including taxol, RIF, RU486, SR12813, clotrimazole, phenobarbital, and hyperforin (Blumberg et al., Genes Dev 12:3195-205 (1998); Kliewer et al., Cell 92:73-82 (1998); Kliewer et al., Endocr Rev 23:687-702 (2002), which are hereby incorporated by reference in their entirety). Thus, the ability of SFN to inhibit ligand-mediated activation of SXR was tested using transfection assays. Two different SXR ligands, RIF and RU486, were able to strongly induce SXR reporter activities in SXR-transfected cells (FIG. 1B). SFN significantly inhibited both RIF and RU486 induced reporter activities. This inhibition was SFN dose-dependent; it inhibited SXR reporter activity at a concentration as low as 1 μM. At 25 μM, SFN blocked most of the RIF induced SXR reporter activity. Dose-response analysis revealed that the IC₅₀ for SFN inhibition of 10 μM RIF-induced CYP3A4 promoter activity was about 12 μM (FIG. 1C). To further confirm that SFN inhibited SXR function, HepG2 cells were transfected with a GAL4 reporter along with a vector expressing the SXR ligand-binding domain linked to the DNA binding domain of GAL4 (GAL4-SXR). Consistent with the results obtained using the full-length SXR, SFN elicited a similar potency of inhibition of GAL4-SXR activity (FIG. 1D), with an IC₅₀ of 14 μM.

To determine whether SFN acts specifically on SXR, the ability of SFN to inhibit ligand activation of a number of other nuclear hormone receptors was evaluated, including mouse PXR (mPXR), rat PXR (rPXR), CAR, VDR, PPARα, and PPARγ. SFN did not inhibit ligand activation of any of these other nuclear hormone receptors (tested at 10 μM concentration), nor did it serve as an activating ligand (FIG. 1E). Surprisingly, although 10 μM SFN can efficiently inhibit SXR activity, it did not inhibit rodent PXR (mPXR or rPXR) activity. This observation is consistent with an in vivo study that found that SFN did not inhibit rat CYP3A gene expression (Hu et al., J Pharmacol Exp Ther 310:263-71(2004), which is hereby incorporated by reference in its entirety). These data suggest that SFN is a species-selective antagonist of human SXR function, perhaps analogous to the known species specificity of RIF as a good human, but not rodent, SXR/PXR ligand (Blumberg et al., Genes Dev 12:3195-205 (1998), which is hereby incorporated by reference in its entirety).

### Example 8

### SFN Can Specifically Bind to SXR

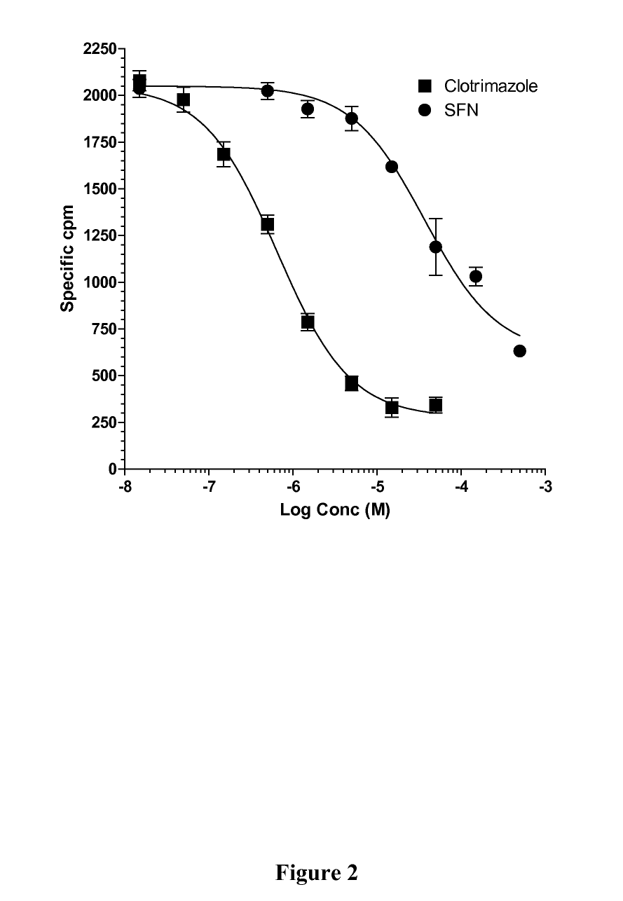

Because SFN effectively inhibited SXR activities in transient transfection assays (FIG. 1), it is likely that SFN works as an antagonist of SXR. Most natural and synthetic nuclear receptor agonists or antagonists exert their effects by directly binding to the nuclear receptor ligand binding domain (LBD). Thus, whether SFN can directly bind to purified SXR protein in vitro was then determined using a sensitive scintillation proximity ligand-binding assay (Tabb et al., J Biol Chem 278, 43919-27 (2003), which is hereby incorporated by reference in its entirety). This assay employed the high-affinity SXR ligand ³H-SR12813 and recombinant histidine-6-tagged-SXR co-expressed with the SRC-1 receptor interacting domain. SFN as well as clotrimazole (positive control) displaced ³H-SR12813 from the SXR LBD in a dose dependent manner (FIG. 2). The Kᵢ for SFN binding to SXR was 16 μM, a value in the range of other known SXR ligands (Jones et al., Mol Endocrinol 14:27-39 (2000), which is hereby incorporated by reference in its entirety). In addition, the affinity was similar to the value obtained for inhibition of SXR function in transfection experiments (FIGS. 1C and D). These results infer that SFN binds specifically to the ligand binding domain of SXR.

### Example 9

### SFN Inhibits SXR Co-Activator Interactions

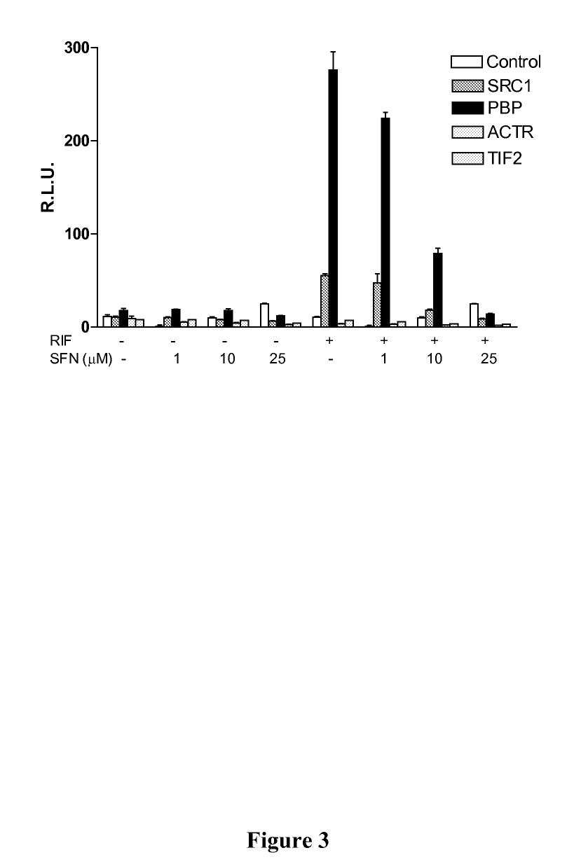

In the absence of ligand, nuclear receptors form a complex with corepressors that inhibit transcriptional activity of the complex through the recruitment of histone deacetylase. When a ligand binds to its nuclear receptor, a conformational change occurs, resulting in dissociation of corepressor and recruitment of coactivator proteins (Glass et al., Genes Dev 14:121-41 (2000); Rosenfeld et al., J Biol Chem 276:36865-8 (2001), which are hereby incorporated by reference in their entirety). Coactivator recruitment is, therefore, a critical part of nuclear receptor signaling pathways. Several coactivators have been shown to be important for nuclear receptor activation, including the steroid receptor coactivator-1 (SRC-1), transcriptional intermediary factor (TIF2), activator of thyroid and retinoic acid receptor (ACTR), and peroxisome proliferator-activated receptor-binding protein (PBP) (Synold et al., Nat Med 7:584-90 (2001); Dussault et al., J Biol Chem 276:33309-12 (2001), which are hereby incorporated by reference in their entirety). Since SFN was shown to block ligand binding to SXR, it was of interest to determine whether SFN also inhibits ligand-induced recruitment of co-activators to SXR. Mammalian two-hybrid assays were used to evaluate whether SFN affects the SXR and co-coactivator interaction. HepG2 cells were transfected with a GAL4 reporter, a vector expressing VP16-SXR, and an expression vector for the GAL4 DNA-binding domain or the GAL4 DNA-binding domain linked to the receptor interaction domains of the indicated co-activators. Consistent with previous reports (Synold et al., Nat Med 7:584-90 (2001); Dussault et al., J Biol Chem 276:33309-12 (2001), which are hereby incorporated by reference in their entirety), RIF strongly promoted the specific interaction of SRC-1 and PBP, but had no significant interaction with ACTR and TIF2 (FIG. 3). SFN inhibited RIF induced SRC-1 and PBP recruitment to SXR in a dose dependent manner. Thus, SFN, although structurally distinct from previously described natural or synthetic SXR ligands, can antagonize its function via direct binding to SXR. Inhibition of ligand binding, and subsequent inhibition of SXR coactivator recruitment, thereby potentially prevents ligand mediated SXR transcriptional activation of SXR-regulated genes in a concentration dependent manner.

### Example 10

### SFN Inhibits SXR-Mediated CYP3A4 Expression in LS180 Cells and Human Primary Hepatocytes

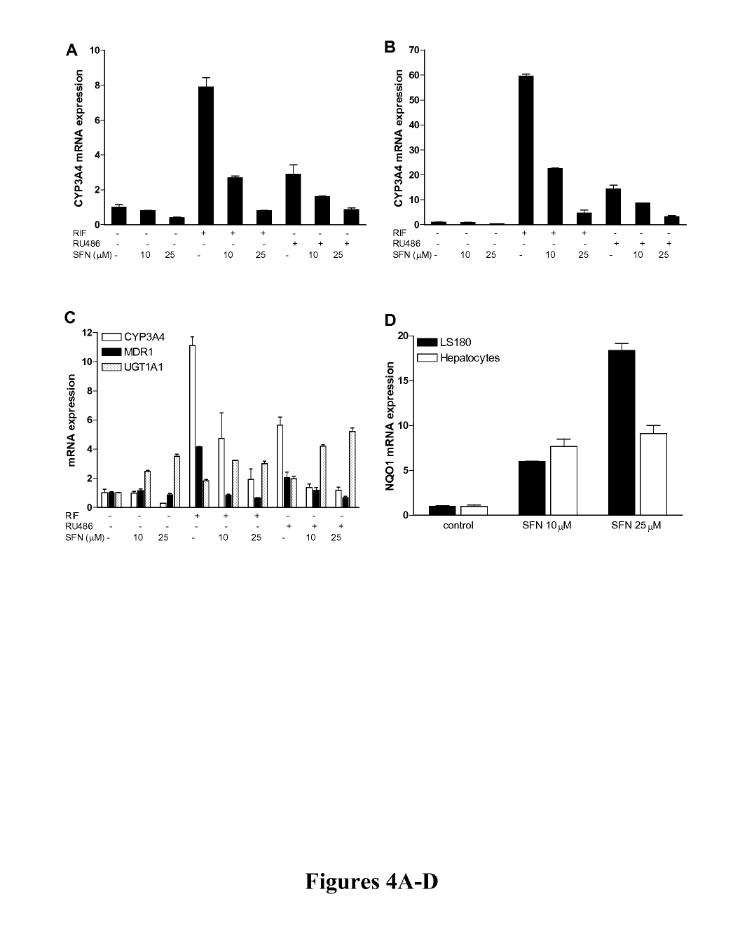

Given the fact that SXR is a major regulator of CYP3A4 and that SFN seems to be an effective antagonist of human SXR, it was evaluated whether SFN modulates SXR-mediated CYP3A4 gene expression. Human intestinal LS180 cells and primary hepatocytes were used for CYP3A4 gene expression analysis. LS180 cells are derived from a human colonic epithelial tumor and represent one of very few human-derived cell lines that have been demonstrated to have functional SXR and inducible CYP3A4 (Synold et al., Nat Med 7:584-90 (2001); Zhou et al., Drug Metab Dispos 32:1075-82 (2001), which are hereby incorporated by reference in their entirety). LS180 cells and human primary hepatocytes from two different donors were pre-treated with various concentrations of SFN for 24 hrs before addition of 10 μM RIF or RU486. Total RNA was isolated 24 hrs later and quantitative RT-PCR (QRT-PCR) was performed to measure CYP3A4 gene expression. As expected, both RIF and RU486 were able to induce CYP3A4 gene expression; RIF was a more potent inducer of CYP3A than RU486, consistent with previous reports (Zhou et al., Drug Metab Dispos 32:1075-82 (2001), which is hereby incorporated by reference in its entirety). SFN caused a dose-related reduction in RIF- and RU486-mediated induction of CYP3A4 in both primary hepatocytes (FIGS. 4A and B) and LS180 cells (FIG. 4C). Similar to the results obtained from transfection experiments (FIG. 1), SFN was able to significantly inhibit both RIF and RU486 induced CYP3A4 expression at a 10 μM concentration and almost completely blocked CYP3A4 induction at 25 μM. Interestingly, SFN also significantly reduced the basal level of CYP3A4 expression in primary hepatocytes, as observed previously (Gross-Steinmeyer et al., Toxicological Sciences 84(S1): 1495 (2005), which is hereby incorporated by reference in its entirety). Furthermore, in LS180 cells, SFN inhibited RIF or RU486 induced expression of MDR1 (ABCB1), a gene that is also regulated by SXR (FIG. 4C). As expected, SFN induced UGT1A1 gene expression, presumably through a SXR-independent and Nrf2-dependent pathway (Basten et al., Carcinogenesis 23:1399-404 (2002), which is hereby incorporated by reference in its entirety). Moreover, SFN significantly induced another Nrf2 target gene, NQO1, in both LS180 cells and human primary hepatocytes (FIG. 4D). The net inductive effect of SFN on UGT1A1 gene expression is intriguing, as the gene is also regulated in part by SXR. Indeed, both RIF and RU486 slightly induced UGT1A1 expression, consistent with a previous studies (Zhou et al., Drug Metab Dispos 32:1075-82 (2001); Xie et al., Proc Natl Acad Sci USA 100:4150-55 (2003), which are hereby incorporated by reference in their entirety). Thus, it is clear that two distinct pathways are involved in SFN mediated gene regulation: SFN activates the Nrf2 signaling pathway and induces ARE target genes such as NQO1 and UGT1A1, but it also acts as an antagonist of SXR, thereby inhibiting SXR-mediated CYP3A4 and MDR1 gene expression.

### Example 11

### SFN Suppresses Constitutive and Inducible CYP3A4-Mediated Midazolam (MDZ) Clearance in Human Primary Hepatocytes

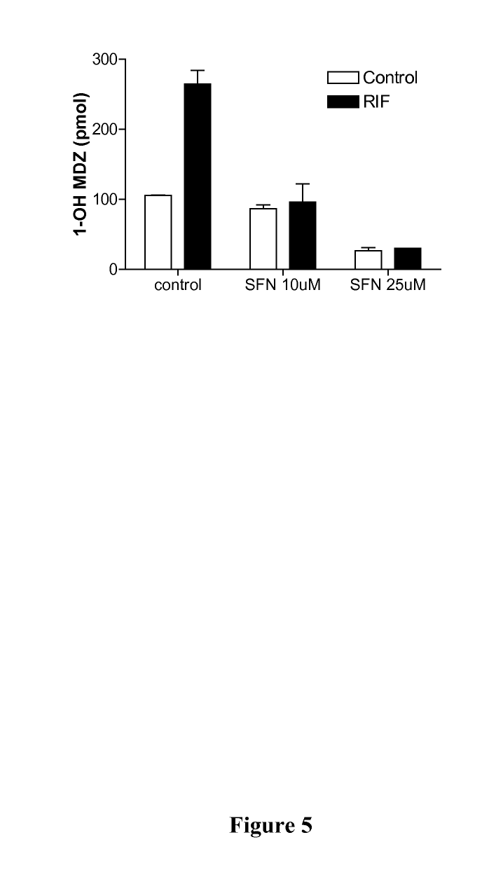

Midazolam is a commonly used short-acting benzodiazepine that is metabolized mainly to 1′-hydoxymidazolam (1′OH-MDZ) and almost exclusively by CYP3A4 (Fabre et al., Biochem Pharmacol 37:4389-97 (1988); Kronbach et al., Mol Pharmacol 36:89-96 (1989), which are hereby incorporated by reference in their entirety). MDZ has been used successfully as an in vivo and in vitro CYP3A4 probe to measure CYP3A4 metabolic activity (Paine et al., Clin Pharmacol Ther 60:14-24 (1996); Paine et al., J Pharmacol Exp Ther 283:1552-62 (1997), which are hereby incorporated by reference in their entirety). It was tested whether SFN suppresses MDZ clearance in human primary hepatocytes. Human primary hepatocytes were pre-treated with 10 or 25 μM SFN for 24 hrs before addition of 10 μM RIF. After 24 hrs, cells were rinsed with PBS and then incubated with 8 μM MDZ for 6 hrs. Supernatant was collected and 1′OH-MDZ concentration was measured by LC-MS. Consistent with CYP3A4 gene expression analysis, RIF effectively induced MDZ clearance about 3-fold, and SFN blocked both basal and RIF induced MDZ clearance in a dose dependent manner (FIG. 5). Interestingly, SFN almost completely suppressed RIF induced MDZ clearance at a 10 μM concentration, whereas it decreased RIF induced CYP3A4 gene expression by only 50% at the same concentration. Previous studies demonstrated that SFN is not a direct inhibitor of CYP3A4 catalytic activity, even at concentrations as high as 50 μM. Therefore, the block of RIF induced MDZ clearance by SFN most likely reflects the inhibition of CYP3A4 expression rather than CYP3A4 enzyme activity.

### Example 12

### Structural Determinants of SXR Antagonism by SFN

There are numerous other naturally occurring isothiocyanates present in a variety of cruciferous vegetables. Since SFN is structurally distinct from previously described natural or synthetic ligands of SXR, naturally-derived phytochemicals that represent structural analogs of SFN were tested to elucidate which part of SFN contributes to its antagonistic effect. The results suggested that the isothiocyanate moiety is critical for antagonism of SXR since the nitrile breakdown product of SFN (SFN-nitrile) had no inhibitory activity. Moreover, the methylsulfoxide part of the molecule also plays a role in SXR antagonism. Replacing the methysulfoxide part with a phenyl (PEITC) resulted in a substantial loss of the inhibitory effect. Interestingly, the oxidation state of the methylsulfide moiety seems to be important as well. A fully reduced sulfur (Erucin) had much less inhibitory activity toward SXR function, and the fully oxidized sulfur, Cheirolin, had similar inhibitory effects with SFN at high concentrations while it was less potent at low concentration. Similar to Cheirolin, shortening the carbon chain from n-butyl to n-propyl (Iberin) had little effect on inhibitory potency, although linker length and rotational flexibility may be important for optimal interaction with SXR. Since all of these compounds are natural products found in varying concentrations in cruciferous vegetables, the results may have practical value in addition to helping to elucidate the structure-function relationship for SFN as an SXR antagonist.

### Example 13

### Hepatocyte Isolation and Culture

Human primary hepatocytes (HPH) were isolated from viable human livers that were rejected for transplantation for various reasons. The isolation was performed at the University of Pittsburgh, PA, USA as described by Strom et al., Methods in Enzymology, 272:388-401 (1996), which is hereby incorporated by reference in its entirety. In culture, HPH were maintained in William's E media supplemented with ITS+ reagent 1x (Collaborative Biochemicals; 6.25 μg/ml insulin, 6.25 μg/ml transferrin, 6.25 μg/ml selenious acid, 1.25 μg/ml bovine serum albumin, and 5.35 μg/ml linoleic acid), 2 mM L-glutamine, 0.1 μM dexamethasone, 100 μg/ml penicillin, 100 U/ml streptomycin, 0.25 μg/ml amphotericin B (Fungizone®), on a rigid collagen substratum overlaid with matrigel (Collaborative Biochemicals). Previous studies have shown optimal responsiveness and expression of biotransformation enzymes under these conditions (Gross-Steinmeyer et al., Xenobiotica 34(7): 619-32 (2005), which is hereby incorporated by reference in its entirety). Treatments with phytochemicals were initiated ≧48 h after seeding.

### Example 14

### SFN Down-Regulates CYP3A4 in a Dose-Depedant Manner

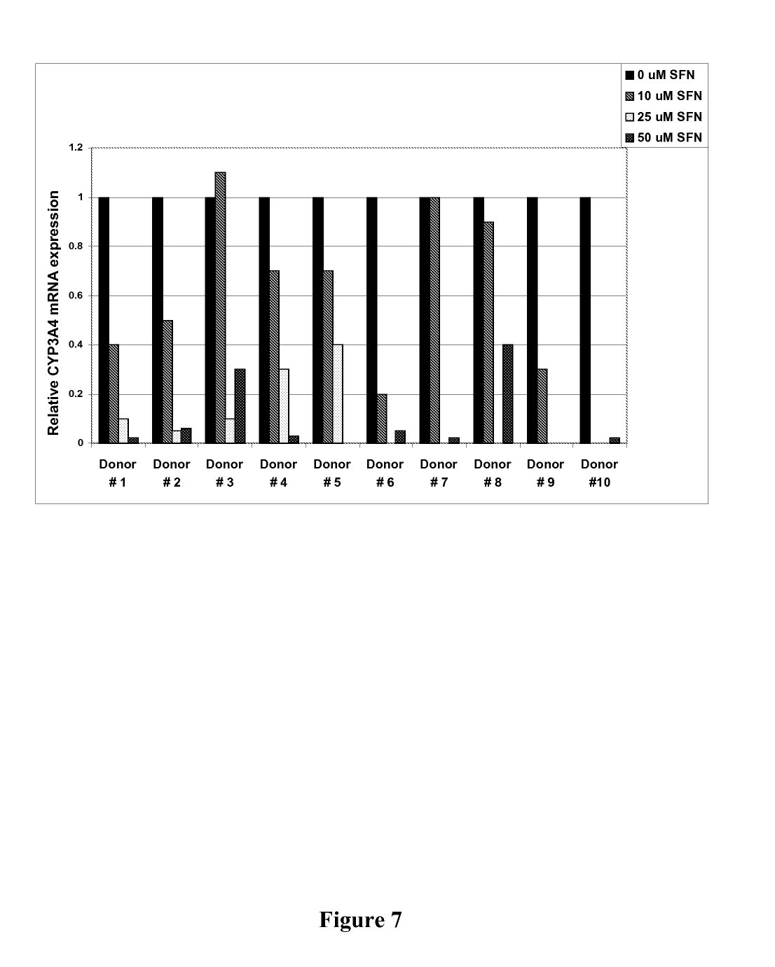

Consistent with the microarray results, SFN showed a drastic down-regulation of CYP3A4 mRNA by Q-RT-PCR. This effect was dose-dependant and highly consistent with all hepatocyte preparations. The average CYP3A4 mRNA levels relative to the solvent control were 69%, 20%, and 13% at 10, 25, and 50 μM SFN, respectively (FIG. 7). However, CYP3A4 responses in single individuals were as low as 2% of the solvent control. An important point to consider in interpreting these results is that CYP3A4 mRNA is normally relatively low at the beginning of human hepatocyte experiments (T0), but continually ‘recovers’ to a much higher level over 48 hrs of incubation (T48). Thus, one must compare the effects of a treatment by looking at how the treatment changed the rate of increase in mRNA over 48 hrs. As the CYP3A4 mRNA level after 48 hr of exposure to the highest dose of SFN was basically unchanged from T0, it can be concluded that SFN inhibits the synthesis of new CYP3A4 message, and/or enhances the rate of CYP3A4 mRNA degradation. SFN dose-dependently and consistently reduced CYP3A4 mRNA levels, even at higher constitutive expression of CYP3A4 beyond 48 h in culture.

### Example 15

### SFN Decreases CYP3A4 Catalyic Activity

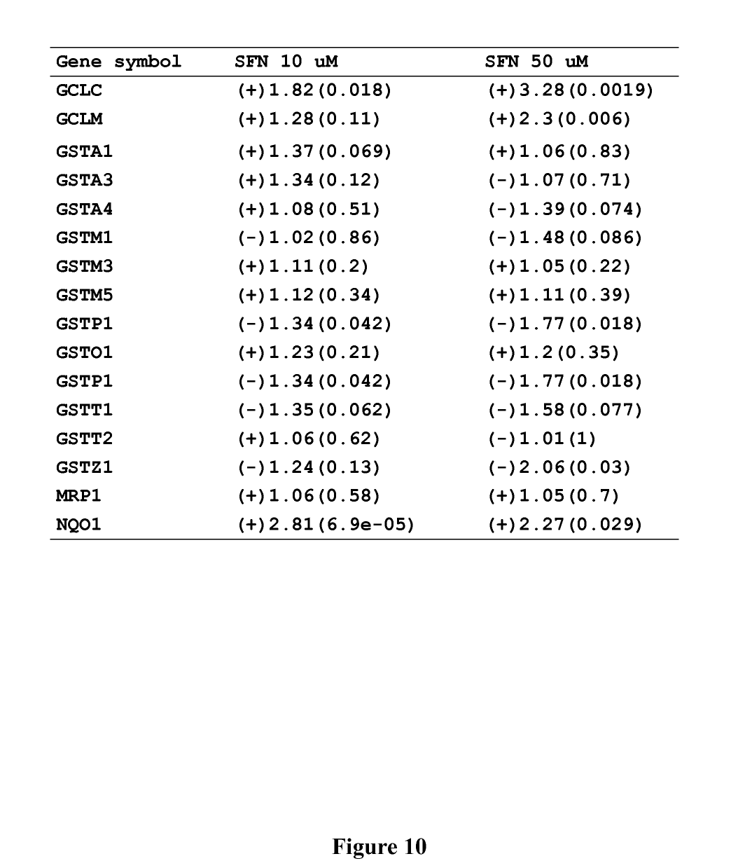

As shown above, SFN, a natural dietary product, is an antagonist of SXR, inhibits SXR-mediated CYP3A4 expression, and the decrease in CYP3A4 mRNA results in a reduction in CYP3A4 catalytic activity. SFN has been promoted as a putative chemopreventive agent to reduce cancer, and it has been demonstrated previously that it provides substantial protection against aflatoxin B1 induced genotoxicity in human primary hepatocytes (Gross-Steinmeyer et al., Toxicological Sciences 80:S1 (2004), which is hereby incorporated by reference in its entirety). It is well established that SFN and other isothiocyanates are effective inducers of phase II detoxification pathways in animal models, and that this is thought to be the primary chemopreventive mechanism. Consistent with animal studies, gene expression profiling of human primary hepatocytes treated with SFN showed increased mRNA levels of several detoxification enzymes including NQO1 and Glutamate Cysteine Ligase (both GCLM and GCLC) (FIG. 10). Most chemical carcinogens require CYP enzyme-mediated metabolic activation before exerting their effects (Conaway et al., Curr Drug Metab 3:233-55 (2002), which is hereby incorporated by reference in its entirety). Here, it has been shown that SFN inhibits SXR transactivation and SXR-mediated CYP3A4 expression which may also contribute to its cancer chemopreventive effects. SFN can efficiently inhibit SXR transactivation by directly binding to SXR and inhibiting coactivator recruitment. Based on current knowledge, SFN is the first effective and relatively non-toxic SXR antagonist ever reported.

### Example 16

### Broccoli Juice Extract Containing SFN Inhibits CYP3A4 Expression and Catalyic Activity in Human Hepatocytes

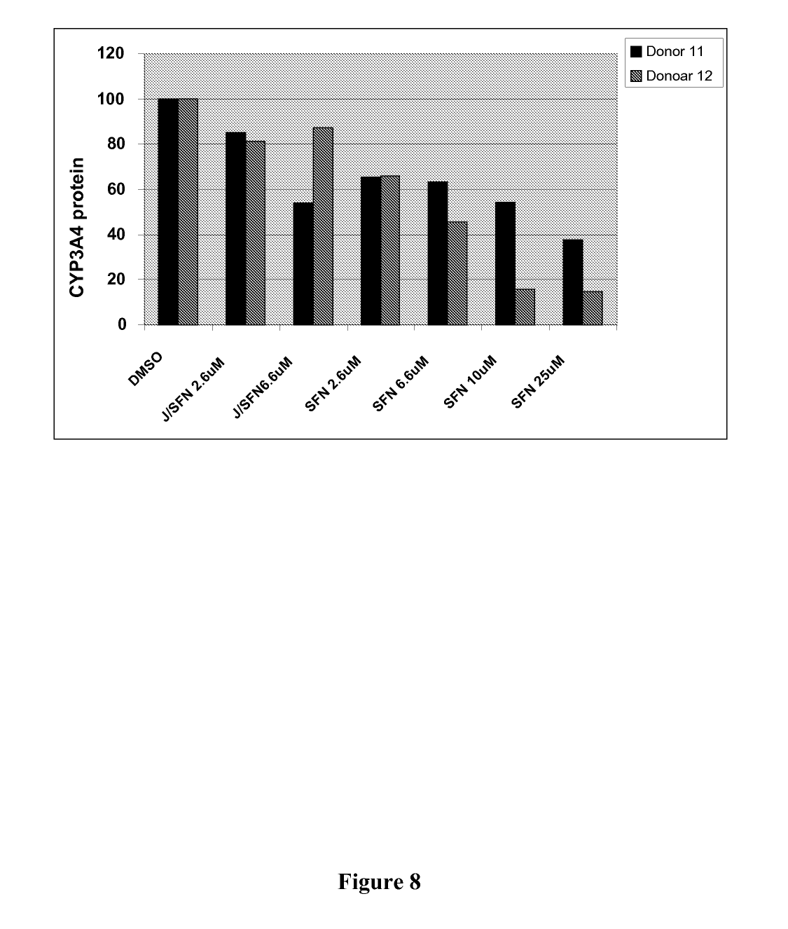

Since SFN is found abundantly in broccoli, SFN present in broccoli was tested to determine whether it is capable of the same effect. Hepatocytes from two donors (#11 and #12) were treated with Broccoli juice extract containing 2.6 uM (J/SFN 2.6 uM) or 6.6 uM SFN (J/SFN 10 uM), or 2.6 uM, 6.6 uM, 10 uM and 25 uM SFN. CYP3A4 protein levels were determined by immunoblot analysis. Broccoli juice extract was analyzed for SFN content, and used in preliminary experiments with two human hepatocyte preparations. FIG. 8 shows that broccoli juice extract containing 2.6 uM and 6.6 uM SFN (final conc) produced an average 20% and 30% decrease in CYP3A4 mRNA, respectively, approximately equivalent to that seen with purified SFN.

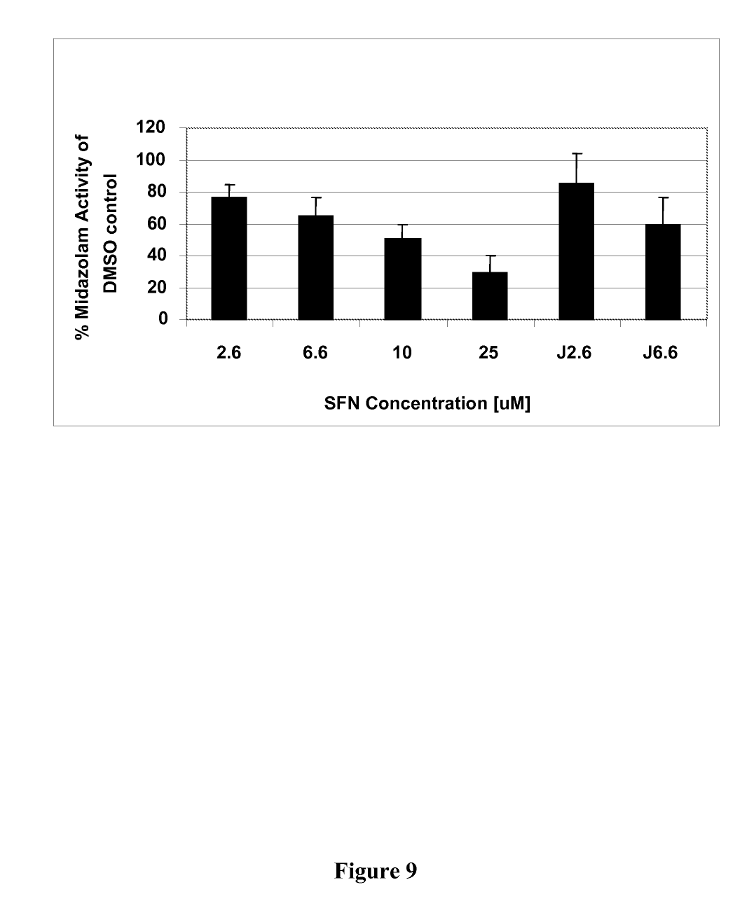

SFN present in broccoli was also demonstrated to decrease CYP3A4 catalytic activity. FIG. 9 depicts primary human hepatocytes from three donors (#11, #12, and #13) that were treated with Broccoli juice extract containing 2.6 μM (J2.6) or 6.6 μM SFN (J6.6), or 2.6 μM, 6.6 μM, 10 μM, and 25 μM SFN. S9 fractions were prepared and midazolam activities were measured as described before. Consistent with the results obtained with SFN (FIG. 5), Broccoli juice inhibited CYP3A4 mediated midazolam clearance in a dose dependent manner.

Because SXR is involved in the regulation of numerous genes, it would be expected that inhibition of SXR function may cause changes in gene expression for many genes. FIG. 10 demonstrates that, although SXR has the most pronounced effect on CYP3A4 regulation, the expression of numerous other genes is modulated in part by SFN. The genes that are ‘up-regulated’ are likely responding to the ability of SFN to activate the Keap1/Nrf2/Antioxidant Response Element Pathway (Yu and Kensler, Mutat Res 591:93-102 (2005), which is hereby incorporated by reference in its entirety) whereas the genes that are ‘down regulated’ are likely responding to the antagonistic effects of SFN on SXR.

SXR is expressed at high levels in the liver and intestine where it acts as a xenobiotic sensor that regulates the expression of cytochrome P450 enzymes such as CYP3A4 and CYP2C8; conjugation enzymes such as, UGT1A1; and ABC family transporters such as, MDR1 and MRP2 (Synold et al., Nat Med 7:584-90 (2001); Dussault et al., J Biol Chem 276:33309-12 (2001), which are hereby incorporated by reference in their entirety). SXR is thus a master regulator of xenobiotic clearance, coordinately controlling steroid and xenobiotic metabolism and transport (Willson et al., Nat Rev Drug Discov 1:259-66 (2002); Xie et al., J Biol Chem 276:37739-42 (2001), which are hereby incorporated by reference in their entirety). This study showed that SFN inhibits SXR function and the expression of its target gene at low micromolar concentrations. SFN is very abundant in broccoli and especially broccoli sprouts, with a reported concentration in broccoli sprouts of about 10 μmol/gm (Shapiro et al., Cancer Epidemiol Biomarkers Prev 10:501-8 (2001), which is hereby incorporated by reference in its entirety). In vitro data suggest that SFN is rapidly absorbed by cells, conjugated efficiently with glutathione (GSH), and excreted mainly as the GSH conjugate (Zhang, Y., Carcinogenesis 21:1175-82 (2000); Zhang et al., Biochem J 364:301-7 (2002), which are hereby incorporated by reference in their entirety). However, the peak plasma concentration of unconjugated SFN in human subjects who ingest SFN in a “broccoli soup” can reach 4-5 μM (Gasper et al., Am J Clin Nutr 82:1283-91 (2005), which is hereby incorporated by reference in its entirety). Therefore, the concentration of SFN used in this study is potentially physiologically relevant, and certainly achievable via pharmacological treatments.

Although SFN is structurally unlike any previously identified class of SXR ligands, it can directly bind to SXR and strongly inhibit SXR coactivator recruitment (FIGS. 2 and 3). Interestingly, compared with human SXR, SFN does not inhibit mouse or rat PXR activities at the same concentration (FIG. 1D). It is known that the induction of hepatic P450 enzymes, especially CYP3A, differs across vertebrate species, and interspecies difference in the pharmacology of SXR/PXR has been identified as the basis for much of this difference (Blumberg et al., Genes Dev 12:3195-205 (1998); LeCluyse, E. L., Chem Biol Interact 134:283-9 (2001); Tabb et al., in Toxicogenomics, eds. Inoue, T. & Pennie, W. D. (Springer-Verlag, Tokyo), pp. 115-125 (2003), which are hereby incorporated by reference in their entirety). There are significant differences in the xenobiotic response between humans and rodents, and these are completely explained by the pharmacology of SXR. For example, the antibiotic rifampicin, the anti-diabetic drug troglitazone and the cholesterol-reducing drug SR12813 were found to be effective activators of both human and rabbit SXR, but had little activity on mouse or rat SXR (Jones et al., Mol Endocrinol 14:27-39 (2000), which is hereby incorporated by reference in its entirety). In contrast, pregnenolone 16a-carbonitrile (PCN) is a more potent activator of rat and mouse SXR than of human or rabbit SXR (Jones et al., Mol Endocrinol 14:27-39 (2000), which is hereby incorporated by reference in its entirety). In addition, some polychlorinated biphenyls (PCBs) have been identified as human SXR agonists, but act as rodent PXR antagonists (Tabb et al., Environ Health Perspect 112:163-9 (2004), which is hereby incorporated by reference in its entirety). The crystal structure of the SXR LBD suggested which amino acid differences in the LBD of SXR contributed to species differences in ligand activation of human SXR and mouse PXR and induction of CYP3A (Watkins et al., Science 292:2329-33 (2001), which is hereby incorporated by reference in its entirety). Further characterization of how SFN differentially interacts with human or rodent SXR/PXR ligand binding domain may explain the species-specific effects of SFN. These experiments show that both ends of the SXR molecule are critical for optimal SXR antagonism. This suggests bipolar anchoring of the relatively small molecule through hydrogen and possibly disulfide bonding with amino acid residues in the ligand binding domain.

Human CYP3A4 is expressed at high, but variable, levels in liver and small intestine and is involved in the metabolism of over 50% of pharmaceutical agents, including several chemotherapeutic drugs. These large interindividual differences in hepatic and intestinal CYP3A4 activity (Thummel et al., Clin Pharmacol Ther 59:491-502 (1996); von Richter et al., Clin Pharmacol Ther 75:172-83 (2004), which are hereby incorporated by reference in their entirety), contribute to difficulties in safe and effective dosing of narrow therapeutic index CYP3A4 substrates. Genetic differences in CYP3A4 or its regulatory genes have not explained much of this variability. Thus, interindividual differences in exposure to dietary or endogenous agents that modulate CYP3A4 transcription may contribute to functional CYP3A4 variability (Blumberg et al., Genes Dev 12:3195-205 (1998), which is hereby incorporated by reference in its entirety). Here, SFN decreased constitutive CYP3A4 mRNA levels and attenuated RIF- and RU486-mediated CYP3A4 induction in human intestinal cells and primary hepatocytes suggest that SFN may be a dietary component affecting inter-individual variability in basal CYP3A4 expression and drug-drug interactions.

Induction or inhibition of CYP3A4 is a common cause of adverse drug-drug interactions. For example, it has been well documented that administration of RIF significantly induces CYP3A4 expression and that this can contribute to adverse drug interactions frequently associated with RIF treatment for tuberculosis. Also, in a study of human volunteers, RIF caused a 95% decrease in the AUC (area under the curve) of the plasma concentration-time curve of orally administered midazolam (Niemi et al., Clin Pharmacokinet 42:819-50 (2003), which is hereby incorporated by reference in its entirety). Oral midazolam, triazolam, simvastatin, verapamil, and most dihydropyridine calcium channel antagonists are ineffective during RIF treatment, because of their excessive clearance by induced hepatic and intestinal levels of CYP3A4. In addition, the plasma concentrations of the antimycotics itraconazole and ketoconazole and the HIV protease inhibitors indinavir, nelfinavir, and saquinavir, are also greatly reduced by rifampicin, potentially resulting in reduced drug efficacy (Niemi et al., Clin Pharmacokinet 42:819-50 (2003), which is hereby incorporated by reference in its entirety). Indeed, the use of RIF with these HIV protease inhibitors is contraindicated to avoid treatment failures. Rifampicin can also cause acute transplant rejection in patients treated with immunosuppressive drugs, such as cyclosporin (Niemi et al., Clin Pharmacokinet 42:819-50 (2003), which is hereby incorporated by reference in its entirety). Although research on the causes of drug interactions has focused primarily on pharmaceutical agents, numerous examples exist where components of the diet modify CYP activity, particularly CYP3A4. For example, St. John's Wort, a widely used herbal antidepressant, is able to interact with a variety of drugs. Hyperforin, the active constituent of St. John's Wort, can induce drug metabolism through activation of SXR and induction of CYP3A4 expression (Wentworth et al., J Endocrinol 166:R11-6 (2000); Moore et al., Proc Natl Acad Sci USA 97:7500-2 (2000), which are hereby incorporated by reference in their entirety). Results from the present study indicate that SFN, a component of the human diet, is able to antagonize SXR activity and SXR-mediated CYP3A4 expression. Because SFN is a natural and relatively non-toxic SXR antagonist, it has the potential to reduce adverse drug responses that arise through the induction of CYP3A4 and other SXR target genes.

In summary, it is shown that SFN is a selective and effective antagonist of SXR function and drug-induced activation of SXR target genes, including CYP3A4. These findings suggest a complementary mechanism by which ingestion of this naturally occurring phytochemical may reduce the risk of certain cancers through a reduction in CYP3A4-mediated reactive metabolite formation. The data also support the potential use of SFN as an adjuvant to prevent CYP3A4 induction and accompanying adverse drug-drug interactions in patients receiving chronic therapy with SXR agonists.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A method of inhibiting CYP3A4 induction, said method comprising:
administering a compound of the following formula:
R₁—X—(CH₂)ₙ—N═C═S
wherein:
R₁ is a C₁ to C₄ alkyl group,
X is S═O or O═S═O, and
n is an integer of 2-5,
that binds to a Pregnane X Receptor or Steroid and Xenobiotic Receptor (SXR or NR1I2) under conditions effective to inhibit CYP3A4 gene induction.

2. The method of claim 1, wherein the compound has the following formula:

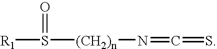

3. The method of claim 2, wherein R₁ is methyl and n is 4.

4. The method of claim 1, wherein said administering is carried out under conditions effective to additionally inhibit induction of a gene selected from the group consisting of ABCB1 (MDR1), ABCC2 (MRP2), CYP1A2, CYP2B6, CYP2C8, CYP2C9, CYP2C19, CYP3A7, CYP7A1, SULT2A1, UGT1A1, UGT1A3, UGT1A4, PAPSS2, ALAS1, and AHR.

5. A method of preventing a loss of efficacy of a drug that is a substrate of CYP3A4 in a subject that is repeatedly administered a CYP3A4 inducer, said method comprising:
administering to the subject a compound of the following formula:
R₁—X—(CH₂)ₙ—N═C═S
wherein:
R₁ is a C₁ to C₄ alkyl group,
X is S═O or O═S═O, and
n is an integer of 2-5,
together with the CYP3A4 inducer under conditions effective to prevent a loss of efficacy of a drug that is a substrate of CYP3A4 in a subject that is repeatedly administered CYP3A4 inducer.

6. The method of claim 5, wherein the compound has the following formula:

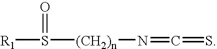

7. The method of claim 6, wherein R₁ is methyl and n is 4.

8. The method of claim 5, wherein the subject is a human.

9. The method of claim 5, wherein said administering is carried out under conditions effective to additionally inhibit induction of a gene selected from the group consisting of ABCB1 (MDR1), ABCC2 (MRP2), CYP1A2, CYP2B6, CYP2C8, CYP2C9, CYP2C19, CYP3A7, CYP7A1, SULT2A1, UGT1A1, UGT1A3, UGT1A4, PAPSS2, ALAS1, and AHR.

10. The method of claim 5, wherein the CYP3A4 inducer is selected from the group consisting of anti-cancer drugs, antibiotics, antiretroviral drugs, an antidepressant, hypolipidemic drugs, anti-hypertensive drugs, anti-inflammatory drugs, anti-epileptic drugs, and wakefulness promoting drugs.

11. The method of claim 10, wherein the CYP3A4 inducer is an anti-cancer drug selected from the group consisting of paclitaxel, topotecan, eptopside, docetaxel, discodermolide, epothilone, vincristine, cyclophosphamide, and tamoxifin.

12. The method of claim 10, wherein the CYP3A4 inducer is an antibiotic selected from the group consisting of rifabutin, rifampicin, flucloxacillin, nafcillin, and artemisinin.

13. The method of claim 10, wherein the CYP3A4 inducer is an antiretroviral drug selected from the group consisting of efavirenz, amprenavir, nevirapine, didanosine, ritonavir, and nelfinavir.

14. The method of claim 10, wherein the CYP3A4 inducer is a hypolipidemic selected from the group consisting of avasimibe and guggulsterone.

15. The method of claim 10, wherein the CYP3A4 inducer is the antidepressant drug hypericin or St. John's Wort.

16. The method of claim 10, wherein the CYP3A4 inducer is an anti-epileptic drug selected from the group consisting of phenytoin, carbamazepine, topiramate, felbamate, and phenobarbital.

17. The method of claim 10, wherein the CYP3A4 inducer is the anti-hypertensive drug bosentan.

18. The method of claim 10, wherein the CYP3A4 inducer is an anti-inflammatory drug selected from the group consisting of dexamethasone, prednisolone, methylprednisolone, or prednisone.

19. The method of claim 10, wherein the CYP3A4 inducer is the wakefulness promoting drug modafanil.

20. A method of preventing a loss of efficacy of a drug that is both a CYP3A4 inducer and CYP3A4 substrate in a subject to whom the drug is repeatedly administered, said method comprising:
administering to the subject being treated with the CYP3A4 inducer and the substrate a compound of the following formula:
R₁—X—(CH₂)ₙ—N═C═S
wherein:
R₁ is a C₁ to C₄ alkyl group,
X is S═O or O═S═O, and
n is an integer of 2-5,
under conditions effective to prevent a loss of efficacy of the drug that is both a CYP3A4 inducer and CYP3A4 substrate in a subject to whom the drug is repeatedly administered.

21. The method of claim 20, wherein the compound has the following formula:

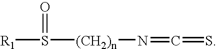

22. The method of claim 21, wherein R₁ is methyl and n is 4.

23. The method of claim 21, wherein the subject is a human.

24. The method of claim 20, wherein the other drug is a ligand for SXR.

25. The method of claim 20, wherein the other drug is a substrate for CYP3A4.

26. The method of claim 20, wherein the other drug is an antiepileptic or psychotropic drug selected from the group consisting of carbamazepine, alprazolam, midazolam, triazolam, buspirone, and ziprasidone.

27. The method of claim 20, wherein the other drug is a hypertensive or cardiovascular drug selected from the group consisting of nifedipine, felodipine, nicaradipine, verapamil, eplerone, lovastatin, atorvastatin, simvastatin, quinidine, and amiodarone.

28. The method of claim 20, wherein the other drug is an antimicrobial drug selected from the group consisting of clarithromycin, erythromycin, itraconazole, ketonazole, quinine, amprenavir, and indinavir.

29. The method of claim 20, wherein the other drug is an anticancer drug selected from the group consisting of etoposide, vinicristine, tamoxifen, toremifene, cyclophosphamide, and ifosfamide.

30. The method of claim 20, wherein the other drug is a pain medication selected from the group consisting of alfentanyl, sufentanyl, and fentanyl.

31. The method of claim 20, wherein the other drug is an immunosuppressive agent selected from the group consisting of cyclosporine, tacrolimus, and sirolimus.

32. The method of claim 20, wherein the other drug is a contraceptive drug selected from the group consisting of ethinyl estradiol (and its prodrug mesantrol), norethindrone, estrone, estradiol, progesterone, medroxyprogesterone, and the antiprogestin mifepristone.

33. The method of claim 20, wherein the other drug is the antiobesity drug sibutramine.

34. The method of claim 20, wherein the other drug is sildenafil.


SEQUENCE LISTING (10 sequences)

SEQ ID NO: 1 - DNA, 26 bp, artificial
  Primer
ggcttcatccaatggactgcataaat

SEQ ID NO: 2 - DNA, 28 bp, artificial
  Primer
tcccaagtataacactctacacagacaa

SEQ ID NO: 3 - DNA, 20 bp, artificial
  Primer
cccatcattgcaatagcagg

SEQ ID NO: 4 - DNA, 23 bp, artificial
  Primer
gagcatacatatgttcaaacttc

SEQ ID NO: 5 - DNA, 20 bp, artificial
  Primer
tgctcattgccttttcacag

SEQ ID NO: 6 - DNA, 20 bp, artificial
  Primer
gggcctagggtaatccttca

SEQ ID NO: 7 - DNA, 21 bp, artificial
  Primer
ggcagaagagcactgatcgta

SEQ ID NO: 8 - DNA, 22 bp, artificial
  Primer
tgatgggattgaagttcatggc

SEQ ID NO: 9 - DNA, 20 bp, artificial
  Primer
ggcctccaaggagtaagacc

SEQ ID NO: 10 - DNA, 20 bp, artificial
  Primer
aggggagattcagtgtggtg